(19) United States
(12) Patent Application Publication
(10) Pub. No.: US 2011/0027776 A1
(43) Pub. Date: Feb. 3, 2011

(54) KITS AND METHODS FOR ASSESSING ANTIOXIDANT REQUIREMENT OF A HUMAN

(76) Inventors: John R. DePhillipo, Margate, NJ (US); Robert P. Ricciardi, Glen Mills, PA (US)

Correspondence Address: THOMPSON HINE L.L.P.;Intellectual Property Group, P.O. BOX 8801, DAYTON, OH, 45401-8801, US

(21) Appl. No.: 11/909,678

(22) PCT Filed: Mar. 28, 2006
(86) PCT No.: PCT/US06/11051

§ 371 Date: Sep. 21, 2010

Related U.S. Application Data
(60) Provisional application No. 60/665,755, filed on Mar. 28, 2005.

(51) Int. Cl. C12Q 1/68; C07C 50/02

(52) U.S. Cl. 435/6; 552/293

(57) ABSTRACT

The invention relates to kits and methods for assessing the desirability of supplementing the diet of a human with reduced coenzyme Q (CoQH₂). The methods involve assessing occurrence in the human's genome of the NQO1*2 polymorphism of the NQO1 gene. Occurrence of a copy of the polymorphism indicates that the human can benefit from dietary supplementation with CoQH₂, and occurrence of two copies (i.e., homozygosity) of the NQO1*2 polymorphism indicates that dietary supplementation with CoQH₂ can be especially desirable.

### BACKGROUND OF THE INVENTION

The invention relates generally to the field of genetic testing and use of antioxidant compositions as dietary supplements.

The properties of molecular oxygen facilitate its utilization in metabolic processes, including in human metabolism. Despite the suitability of oxygen as a substrate for metabolism, oxygen also exists in toxic forms which can damage or kill human cells. Some toxic forms of oxygen form spontaneously in biological systems, and others are formed by operation of metabolic processes in human tissues. Antioxidant compositions are normally present in human tissues and prevent much oxidative damage to tissues. Coenzyme Q (CoQ; sometimes designated ubiquinone) is a component of the electron transport chain in mitochondria, and has also been recognized to act as an antioxidant in human tissues.

CoQ can exist in an oxidized form and a reduced form (designated CoQH₂, ubiquinol, or the hydroquinone form of CoQ). Upon acquisition of a pair of electrons, the oxidized form of CoQ is transformed into CoQH₂. In the CoQH₂ form, CoQ is an effective membrane-soluble antioxidant. CoQH₂ can be converted back to oxidized CoQ by transfer of a pair of electrons to another substrate, such as a toxic form of oxygen or an inappropriately oxidized cellular component. This electron transfer is the basis of the antioxidant action of CoQ. However, oxidized CoQ does not exhibit substantial further antioxidant activity until and unless it is re-converted to the reduced CoQH₂ form.

Transfer of electrons to CoQ to regenerate the antioxidant CoQH₂ can occur by a number of pathways, including a two-electron transfer catalyzed by the mitochondrial enzyme designated DT-diaphorase, which is also known by the names menadione reductase and NAD(P)H:quinone acceptor reductase (Beyer et al., 1994, Molec. Aspects Med. 15(Supp.):s117-s129; Beyer et al., 1996, Proc. Natl. Acad. Sci. USA 93:2528-2532). This enzyme is encoded by the NQO1 gene, and its expression has been recognized as being up-regulated in tissues in which antioxidant activity is necessary (Ross et al., 2000, Chemico-Biological Interactions 129:77-97; Raina et al., 1999, Redox Rep. 4(1-2):23-27; SantaCruz et al., 2004, Neurobiol. Aging 25(1):63-69). Ross et al. disclosed a polymorphism in NQO1 that substantially abolished NQO1 protein expression and activity in homozygous polymorphic transfectant cells. Occurrence of this polymorphism, designated the NQO1*2 polymorphism, has been associated by others with increased risk of developing several types of tumors and with increased benzene-induced hemotoxicity. However, it is believed that no others have recognized a role for using the NQO1*2 polymorphism to select an appropriate antioxidant for a human.

Most, if not all, human genes occur in a variety of forms which differ in at least minor ways. Heterogeneity in human genes is believed to have arisen, in part, from minor, non-fatal mutations that have occurred in the genome over time. In some instances, differences between alternative forms of a gene are manifested as differences in the amino acid sequence of a protein encoded by the gene. Some amino acid sequence differences can alter the reactivity or substrate specificity of the protein. Differences between alternative forms of a gene can also affect the degree to which (if at all) the gene is expressed. However, many heterogeneities that occur in human genes appear not to be correlated with any particular phenotype. Known heterogeneities include, for example, single nucleotide polymorphisms (i.e., alternative forms of a gene having a difference at a single nucleotide residue). Other known polymorphic forms include those in which the sequence of larger (e.g., 2-1000 residues) portions of a gene exhibits numerous sequence differences and those which differ by the presence or absence of portion of a gene.

Numerous disorders and physiological states have been correlated with occurrence of one or more alternative forms of an individual gene in the genome of a human who exhibits the disorder or physiological state. For example, Kimura et al. (2000, Am. J. Opthalmol. 130:769-773) discloses an association between occurrence of a SNP of the manganese superoxide dismutase gene and a form of macular degeneration.

Associations between individual disorders and individual genetic polymorphisms are known. However, disorders can usually result from polymorphisms in any of a relatively large number of genes, and as a result, assessing the polymorphic form(s) of any single gene that occur in a human's genome is usually not predictive of the overall likelihood that the human will develop the disorder.

Many disorders, including many that can be prevented, inhibited, delayed, or reduced in severity by timely consumption of appropriate antioxidant compositions, develop over time. Such compositions are often not consumed, owing to the expense or inconvenience of obtaining the compositions and regularly administering them. Failure of individuals to recognize that their genetic composition predisposes them to certain disorders or renders them less able to benefit from certain antioxidant compositions than others also inhibits effective preventive and therapeutic use of antioxidant compositions.

CoQ is available commercially in the form of dietary supplements. Most CoQ supplements provide CoQ in its oxidized (ubiquinone) form. Supplements containing reduced CoQ have been described (e.g., international patent publication WO 01/52822, U.S. Pat. No. 6,056,971; U.S. Pat. No. 6,300,377; and U.S. Pat. No. 6,441,050), and are available commercially. CoQH₂-containing supplements are recognized for enhanced availability and uptake of CoQH₂, relative to CoQ. However, it is believed that there was no recognition by others of particular groups of individuals who might benefit from such supplements.

A need remains for a method of assessing the antioxidant requirements for a person, based on that person's genetic composition. The invention satisfies this need.

### BRIEF SUMMARY OF THE INVENTION

The invention includes a method of assessing the desirability of supplementing the diet of a human with CoQH₂. The method comprises assessing occurrence in the human's genome of the NQO1*2 polymorphism. Occurrence of a copy of the polymorphism is an indication that it is more desirable to supplement the human's diet with CoQH₂ than that of a human whose genome does not comprise the polymorphism.

### DETAILED DESCRIPTION OF THE INVENTION

The invention relates to the discovery that the NQO1*2 polymorphism can be used an indicator of a person's need for dietary supplementation with reduced coenzyme Q (CoQH₂). By assessing whether a person has no, one, or two copies of the NQO1*2 polymorphism in his or her genome, one can determine whether that person requires dietary supplementation with CoQH₂ and, if such supplementation is deemed necessary, the relative degree of supplementation that is desirable.

In essence, the invention relates to a method of assessing the need of an individual for dietary supplementation with CoQH₂. The method includes analyzing occurrence of the NQO1*2 polymorphism in the individual's genome. If the NQO1*2 polymorphism does not occur in the individual's genome, then the individual does not require supplementation with CoQH₂, and any CoQ supplementation of the individual's diet can be achieved using the oxidized form of CoQ, which can be more readily available and less expensive than CoQH₂. Occurrence of one copy of the NQO1*2 polymorphism in the individual's genome indicates that the individual can benefit from administration of CoQH₂, and that administration of CoQH₂ may be especially recommended for the individual when conditions of increased oxidative stress (e.g., vigorous exercise) are anticipated. If the individual is homozygous for the NQO1*2 polymorphism, then this is an indication that dietary supplementation with CoQH₂ is likely to be effective to achieve an antioxidant effect in the individual, and that dietary supplementation with (oxidized) CoQ is likely to be much less effective for that individual.

### DEFINITIONS

As used herein, each of the following terms has the meaning associated with it in this section.

A “characteristic residue” of a polymorphism is a nucleotide residue, the identity of which is known to vary among the alternative forms corresponding to the polymorphism.

A “molecular beacon oligonucleotide” is a single-stranded oligonucleotides having a fluorescent label (e.g., rhodamine, FAM, TET, VIC, JOE, or HEX) attached to the 5′-end thereof and a fluorescence quencher (e.g., TAMRA or DABCYL) attached to the 3′-end thereof (or vice versa), as described (Kostrikis et al., 1998, Science 279:1228-1229).

Two molecular beacon oligonucleotides are “spectrally distinct” if they can be differentially detected using spectrophotometric or spectrofluorometric methods. Examples of characteristics that can be used to differentiate spectrally distinct oligonucleotides include absorption or excitation wavelength, emission wavelength, and fluorescent lifetime.

An “instructional material” is a publication, a recording, a diagram, or any other medium of expression which can be used to communicate how to use a kit described herein, numerical values for weighting the significance of various polymorphisms that are detectable using the kit, or both. The instructional material of the kit of the invention can, for example, be affixed to a container which contains a kit of the invention or be shipped together with a container which contains the kit. Alternatively, the instructional material can be shipped separately from the container with the intention that the instructional material and the kit be used cooperatively by the recipient.

The “stringency” with which two polynucleotides anneal means the relative likelihood that the polynucleotides will anneal in a solution as the conditions of the solution become less favorable for annealing. Examples of stringent conditions are known in the art and can be found in available references (e.g., Current Protocols in Molecular Biology, John Wiley & Sons, N.Y., 1989, 6.3.1-6.3.6). Aqueous and non-aqueous annealing methods are described in that reference and either can be used. In general, a first pair of polynucleotides anneal with higher stringency than a second pair if the first pair is more likely to anneal (or remain annealed) as one or more of the salt concentration, temperature, and detergent concentration are increased.

A “non-extendable” nucleotide residue is a nucleotide residue that is capable of being added to a polynucleotide by a polymerase (i.e., by extension of the polynucleotide in association with a complement thereof, catalyzed by the polymerase) and that, upon addition to the polynucleotide, renders the polynucleotide incapable of being further extended by the polymerase.

“Coenzyme Q” (CoQ) is a class of lipid-soluble benzoquinones that are known in the art as components of electron transport chains and as antioxidant compounds. CoQ exists in the form of an aromatic quinone “head” and a “tail” of multiple linked isoprene units. CoQ₁₀, which is the primary naturally-occurring form of CoQ has a tail consisting of 10 linked isoprene units. As used herein, CoQ includes all CoQ compounds known in the art for dietary supplementation, not just CoQ₁₀.

### DETAILED DESCRIPTION

The invention relates to the discovery that the NQO1*2 polymorphism can be used an indicator of a person's need for dietary supplementation with reduced coenzyme Q (CoQH₂).

The invention includes a method of assessing the desirability of supplementing the diet of a human with CoQH₂. The method comprises assessing occurrence in the human's genome of the NQO1*2 polymorphism. Occurrence of a copy of the polymorphism is an indication that it is more desirable to supplement the human's diet with CoQH₂ than that of a human whose genome does not comprise the polymorphism. Occurrence of two copies of the polymorphism indicates that is more important to supplement the human's diet with CoQH₂ than it is to supplement the diet of a human in whose genome either one copy or no copies of the NQO1*2 polymorphism occurs. Indeed, because an individual who is homozygous for the NQO1*2 polymorphism will exhibit little or no mitochondrial DT-diaphorase activity, dietary supplementation with CoQH₂ can inhibit, delay, or prevent development of an oxidative stress-related disorder or lessen the severity of any such disorder that develops. Numerous oxidative stress-related disorders are known, including such examples as Alzheimer's disease, macular degeneration, and diabetes.

Human bodies are believed to contain approximately 2 grams of CoQ, and daily turnover is believed to be approximately 0.5 gram per day (Ely et al. 2000, J. Orthomolec. Med. 15(2):63-68). CoQ can be obtained by physiological synthesis or from the diet. Because biosynthesis of CoQ declines as one ages and because the average CoQ content of a typical Western diet is relatively low (i.e., ca. 5 milligrams per day), dietary supplementation is often advisable. CoQ supplements are commonly available in unit dosage forms containing 50, 100, and 200 milligrams per dose. Dosing with 400 milligrams, 800 milligrams, or even greater quantities is known. An ordinarily skilled physician will be able to determine an appropriate daily dosage of CoQ for an individual, taking into account the individual's age, weight, lifestyle, disease state, and the information disclosed herein.

Dietary supplementation with CoQ has been widely disclosed. Most CoQ-containing dietary supplements contain CoQ in its oxidized form, which is more stable and generally less expensive to obtain than the reduced form, CoQH₂. CoQH₂ has been reported to exhibit greater bioavailability than the oxidized form of CoQ (international patent publication WO 01/52822). However, there has been no description by others of differentiation among humans based on their ability to reduce oxidized CoQ.

As described herein, humans in whose genome two copies of the NQO1*2 polymorphism occur will derive greater antioxidant benefit from a dietary supplement containing CoQH₂ than will humans whose genomes do not include a copy of the polymorphism. Likewise, humans in whose genome two copies of the NQO1*2 polymorphism occur will derive greater antioxidant benefit from a dietary supplement containing CoQH₂ than will humans whose genomes include a single copy of the polymorphism. Although the difference between NQO1*2 homozygotes and NQO1*1 homozygotes is likely to be greater than the difference between NQO1*2 homozygotes and NQO1*2/NQO1*1 heterozygotes, the NQO1*2 homozygotes should also derive greater antioxidant benefit from a dietary supplement containing CoQH₂ than the heterozygotes.

By way of example, in a 21-year-old human with no known propensity for oxidative stress-related disorders, it can be suitable to recommend that any CoQ supplement taken by the individual need not contain any CoQH₂ if the individual is homozygous for the (normal) NQO1*1 form of the NQO1 gene. However, if the same individual were found to be a NQO1*2/NQO1*1 heterozygote, then it could be recommended that some or all CoQ taken by the individual as a supplement (e.g., 50 milligrams per day) be in the form of CoQH₂. If this individual were found, using the methods described herein, to be a NQO1*2 homozygote, then it can be recommended that substantially all CoQ taken by the individual as a supplement (e.g., 500 milligrams per day) be in the reduced form.

Occurrence of the NQO1*2 polymorphism can be assessed by substantially any known method of polymorphism detection. Such methods include, by way of example, sequencing-based methods, hybridization-based methods, and primer extension methods (including at least single-base extension methods and PCR amplification methods). The precise method used to detect the polymorphism is not critical, so long as the method is capable of differentiating occurrence of an NQO1*2 polymorphism in a genome from lack of such occurrence. In one embodiment, a nucleic acid derived from an individual's genome is contacted with a first oligonucleotide that anneals with higher stringency with the NQO1*2 polymorphism than with the NQO1*1 form of the NQO1 gene. Annealing of the first oligonucleotide and the nucleic acid is thereafter assessed, with annealing of the first oligonucleotide and the nucleic acid being an indication that the individual's genome comprises the NQO1*2 polymorphism.

The genome of an individual can also be assessed to determine whether the individual's genome includes a normal copy of the NQO1 gene (i.e., the NQO1*1 form of the gene). This assessment can be used to determine whether the allele content of the individual with regard to isoforms of the NQO1 gene. Occurrence of the NQO1*3 polymorphism can be assessed as well. However, because the NQO1*3 polymorphic form is so rare, it can be effectively ignored. Of course, multiple tests can be conducted on an individual's genome (i.e., either as discrete tests or in a single test using multiple probes or primers) to detect multiple NQO1 polymorphisms. Using such a test, one can determine both occurrence of one or more null NQO1 polymorphisms (i.e., either or both of NQO1*2 and NQO1*3) in an individual's genome and whether the individual is homozygous or heterozygous for the disorder-associated polymorphism. This test also permits ‘checking’ of results, since it can both account for all known polymorphic forms and indicate when a previously uncharacterized polymorphism occurs at or near the site of a known polymorphism.

In one embodiment, a pair of oligonucleotide primers are used to amplify a portion of the NQO1 gene that includes a polymorphic region. Detection of one or more of the polymorphisms that occur at the polymorphic region can be achieved by contacting the amplified portion with an oligonucleotide having a sequence that it will anneal under stringent conditions with the amplified portion only if one polymorphism occurs at the portion, but will not anneal with the amplified portion if another polymorphism occurs at that portion. Various acceptable stringent conditions are known in the art, and can be modified by the skilled artisan as appropriate to any particular amplified portion/oligonucleotide pair. An example of stringent conditions is hybridization in 6× sodium chloride/sodium citrate (SSC) at about 45° C., followed by one or more washes in 0.2×SSC, 0.1% (w/v) SDS at 50° C.

In an alternative embodiment, one or more molecular beacon oligonucleotides are used to detect polymorphisms (NQO1*1, NQO1*2, NQO1*3, or some combination of these) in a sample that contains a copy of the subject's genome, a fraction of the subject's genome, or amplification products generated from the subject's genome (e.g., an amplified portion of the NQO1 gene).

Molecular beacon probes are single-stranded oligonucleotides having a fluorescent label (e.g. rhodamine, FAM, TET, VIC, JOE, or HEX) attached to the 5′-end thereof and a fluorescence quencher (e.g. TAMRA or DABCYL) attached to the 3′-end thereof (or vice versa), as described (Kostrikis et al., 1998, Science 279:1228-1229). The sequence of each molecular beacon probe is selected to include two complementary hairpin regions, whereby the probe can self-anneal to form a hairpin structure. The 5′- and 3′-ends are brought into close association when the hairpin structure forms. The probe also comprises a targeting portion which is selected to be complementary to a target sequence (e.g. a single polymorphism of a gene disclosed herein). The targeting portion and at least one of the hairpin regions are located in close proximity to one another, meaning that the targeting portion either overlaps the hairpin region or flanks it, having no more than about 5 nucleotide residues therebetween.

If the hairpin regions of the molecular beacon probe anneal with one another, then the probe does not fluoresce, because the hairpin structure forms and the fluorescence quencher attached to one end of the probe quenches fluorescence of the label attached to the other end of the probe. If the targeting portion of the probe anneals with a region of a nucleic acid having the target sequence, then formation of the hairpin structure is inhibited, the fluorescence quencher is not brought into association with the fluorescent label, and the probe fluoresces. Multiple molecular beacon probes can be used in a single reaction mixture, and fluorescence associated with the probes can be differentiated if the molecular beacon probes are spectrally distinct.

Thus, in this embodiment, one or more molecular beacon probes are used, each having targeting portion which is complementary to a target region (e.g. 20 to 40 nucleotide residues, more preferably 20 to 30 residues) of one polymorphism of the NQO1 gene. The target region includes, and preferably is approximately centered around, the nucleotide residue at which the polymorphism occurs. More preferably, two such probes are used, one having a targeting region completely complementary to the target region of one polymorphism of the gene (e.g., the NQO1*1 form), and the other having a targeting region completely complementary to the target region of another polymorphism of the gene (e.g., the NQO1*2 polymorphism).

In yet another embodiment of how polymorphisms in the NQO1 gene can be assessed, oligonucleotide primers which are complementary to a region adjacent a characteristic residue of a polymorphic form of NQO1 (e.g., residue 609 for the NQO1*2 form or residue 465 for the NQO1*3 form) are extended using a polymerase enzyme, and the identity of the nucleotide residue that is added to the primer in the position complementary to the characteristic residue is determined. The primer can be extended in the presence of non-extendable nucleotide residues in order to ensure that a limited number of (or only one) nucleotide residues are incorporated into the primer. Methods of this type are known in the art (e.g., the SNP-IT® technology of Orchid Biocomputer, Inc.) and are described, for example in U.S. Pat. Nos. 6,013,431 and 6,004,744.

Many tests and test formats are commercially available for detection of polymorphic forms of genes. The format of the test used to detect, distinguish, or detect and distinguish NQO1 polymorphisms is not critical. Rapid tests, including those in which a reagent for detection of one or more polymorphs is fixed to a support can be preferred when relatively rapid turnaround between collection of a genomic sample and reporting of results is desired.

The polymorphic forms of the NQO1 gene described herein are as follows. The normal form of the gene is designated as polymorphic form NQO1*1, and is the form of the gene described in Jaiswal et al., 1988, J. Biol. Chem. 263(27):13572-13578, in Ross, 2004, Atlas Genet. Cytogenet. Oncol. Haematol., ID # NQO1ID375, and elsewhere in the literature. The NQO1*2 polymorphism differs from NQO1*1 in that nucleotide residue 609 is changed from C (in NQO1*1) to T (in NQO1*2), resulting in a change at amino acid residue 187 from proline (in NQO1*1) to serine (in NQO1*2). The NQO1*3 polymorphism differs from NQO1*1 in that nucleotide residue 465 is changed from C (in NQO1*1) to T (in NQO1*3), resulting in a change at amino acid residue 139 from arginine (in NQO1*1) to tryptophan (in NQO1*3). The NQO1*3 polymorphism appears to be very rare—occurring in fewer than 1 in about 20,000 individuals.

Using the information generated from the NQO1 polymorphism-detecting tests described herein, CoQ-containing antioxidant compositions can be formulated for administration to individual human, based on the individual's genome. If the human's genome includes even one NQO1*2 polymorphism (or an NQO1*3 polymorphism), the individual can be expected to have impaired ability to reduce oxidized CoQ. This is an indication that the individual will benefit from administration of CoQH₂. Occurrence of two null NQO1 polymorphisms in the individual's genome is an indication that the individual will exhibit severely impaired ability to reduce oxidized CoQ. For such individuals, it can be recommended that most or all of the CoQ in the supplement formulated for the individual be present in the form of CoQH₂. Furthermore, because the individual can have difficulty reducing CoQ already present in the individual's body, it can be advantageous to administer amounts of CoQH₂ in excess of the amount desired for supplementation purposes, particularly if the individual has (for other reasons than NQO1 polymorphism) heightened susceptibility to an oxidative stress-related disorder, is afflicted with such a disorder, or expects to engage in an activity associated with oxidative stress (e.g., intense exercise) in the near future.

### EXAMPLES

The invention is now described with reference to the following Examples. These Examples are provided for the purpose of illustration only, and the invention is not limited to these Examples, but rather encompasses all variations which are evident as a result of the teaching provided herein.

### Example 1

### Correlation of NQO1 Genotype and CoQ Redox Ratio

A correlation has been discovered between the allele content of the NQO1 gene of human patients and the fraction of CoQ that is present in its reduced form (CoQH₂) in the blood of patients.

In a blinded study, the NQO1 genotype of human subjects was assessed. For two weeks, the subjects did not consume CoQ supplements or any other antioxidant-containing dietary supplement. After that two week period, blood samples were taken from each subject, and the amounts of CoQ and CoQH₂ in each blood sample were determined.

Subjects whose genomes included two copies of the normal (NQO1*1) form of the NQO1 gene exhibited a CoQ redox ratio (concentration of reduced CoQ divided by concentration of oxidized CoQ) of 16.9±2.2. Subjects whose genomes included one copy of NQO1*1 and one copy of NQO1*2 had a CoQ redox ratio of 11.9±1.1. These results demonstrate that NQO1 genotype can be correlated with the redox ratio of CoQ in a human subject.

According to the Human Genome Epidemiology Network database, the NQO1*2 polymorphism occurs in about 40% of Caucasians in the form of heterozygotes (i.e., NQO1*1/NQO1*2 heterozygotes), and in about 5% of Caucasians in the form of homozygotes (i.e., NQO1*2/NQO1*2 homozygotes). These observations suggest widespread applicability for the methods described herein in human populations. In view of the importance of antioxidant activity of CoQ and the DT-diaphorase in cardiac, neuronal, and other tissues, the methods described herein can be expected to be useful for identifying human subjects who will benefit from consumption of dietary supplements containing CoQH₂, and especially for subjects afflicted with or at a risk for developing oxidative stress-related disorders of cardiac, neuronal, and other tissues. Examples of such disorder include Alzheimer's disease, macular degeneration, metabolic syndrome, and diabetes

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A method of assessing the desirability of supplementing the diet of a human with reduced coenzyme Q (CoQH₂), the method comprising assessing occurrence in the human's genome of the NQO1*2 polymorphism, whereby occurrence of a copy of the polymorphism is an indication that it is more desirable to supplement the human's diet with CoQH₂ than that of a human whose genome does not comprise the polymorphism.

2. The method of claim 1, further comprising a suitable dosage of CoQH₂ for supplementation of the human's diet.

3. The method of claim 2, wherein the suitable dosage is substantially no CoQH₂ if the human's genome does not comprise the polymorphism.

4. The method of claim 2, wherein the suitable dosage is at least about 50 milligrams per day of CoQH₂ if the human's genome comprises one copy of the polymorphism.

5. The method of claim 2, wherein the suitable dosage is at least about 500 milligrams per day of CoQH₂ if the human's genome comprises two copies of the polymorphism.

6. The method of claim 1, wherein occurrence of the NQO1*2 polymorphism is assessed by contacting a nucleic acid derived from the human's genome with a first oligonucleotide that anneals with higher stringency with the NQO1*2 polymorphism than with the NQO1*1 form of the NQO1 gene and assessing annealing of the first oligonucleotide and the nucleic acid, whereby annealing of the first oligonucleotide and the nucleic acid is an indication that the human's genome comprises the NQO1*2 polymorphism.

7. The method of claim 6, wherein the first oligonucleotide is attached to a support.

8. The method of claim 6, wherein the first oligonucleotide is a molecular beacon oligonucleotide.

9. The method of claim 6, wherein occurrence of the NQO1*2 polymorphism is further assessed by contacting the nucleic acid with a second oligonucleotide that anneals with higher stringency with the NQO1*1 form of the NQO1 gene than with the NQO1*2 polymorphism and assessing annealing of the second oligonucleotide and the nucleic acid, whereby annealing of the second oligonucleotide and the nucleic acid is an indication that at least one allele of the NQO1 gene in the human's genome does not comprise the NQO1*2 polymorphism.

10. The method of claim 9, wherein the second oligonucleotide is attached to a support.

11. The method of claim 10, wherein the first and second oligonucleotides are attached to the same support.

12. The method of claim 9, wherein the second oligonucleotide is a molecular beacon oligonucleotide.

13. The method of claim 12, wherein the first and second oligonucleotides are spectrally distinct molecular beacon oligonucleotides.

14. A method of formulating a coenzyme Q-(CoQ-)containing antioxidant composition for administration to a human, the method comprising assessing occurrence in the human's genome of the NQO1*2 polymorphism and including CoQH₂ in the composition if the polymorphism occurs in the genome.

15. The method of claim 14, further comprising formulating the composition so that substantially all of the CoQ is in the form of CoQH₂ if the human is homozygous for the polymorphism.

16. A method of assessing the advisability that a human should employ a dietary supplement comprising CoQH₂, the method comprising assessing occurrence in the human's genome of the NQO1*2 polymorphism, whereby occurrence of a copy of the polymorphism is an indication that the human should employ a dietary supplement comprising CoQH₂.

17. A method of assessing the advisability that a human should employ a dietary supplement comprising CoQH₂, the method comprising assessing occurrence in the human's genome of the NQO1*2 polymorphism, whereby homozygosity of the human for the polymorphism is an indication that the human should employ a dietary supplement comprising CoQH₂.

18. A method of assessing the desirability of supplementing the diet of a human with reduced coenzyme Q (CoQH₂), the method comprising assessing occurrence in the human's genome of a null NQO1 polymorphism, whereby occurrence of a copy of the null polymorphism is an indication that it is more desirable to supplement the human's diet with CoQH₂ than that of a human whose genome does not comprise the null polymorphism.

19. The method of claim 18, wherein the null polymorphism is NQO1*2.

20. The method of claim 18, wherein the null polymorphism is NQO1*3.
